(19) United States
(12) Patent Application Publication
(10) Pub. No.: US 2012/0259180 A1
(43) Pub. Date: Oct. 11, 2012

(54) HYDRATION AND NUTRITION SYSTEM

(76) Inventor: Michael Rock, Deerfield, IL (US)

(21) Appl. No.: 13/444,790

(22) Filed: Apr. 11, 2012

Related U.S. Application Data
(60) Provisional application No. 61/474,250, filed on Apr. 11, 2011.

(51) Int. Cl. A61B 5/00

(52) U.S. Cl. 600/300

(57) ABSTRACT

A hydration and nutrition system for monitoring consumption of fluid by a user includes: a reservoir adapted to hold a quantity of fluid; a tube adapted to convey the fluid from the reservoir to the user for consumption; a flow meter adapted to measure and collect data regarding the flow of fluid from the reservoir through the tube and communicate the data regarding the flow of fluid to a computerized nutritional calculator, wherein the computerized nutritional calculator is adapted to calculate a deficit or surplus of fluid consumed as compared to a consumption plan.

### CROSS-REFERENCE TO RELATED APPLICATIONS

This application incorporates by reference and claims priority to U.S. Provisional Patent Application No. 61/474,250 filed Apr. 11, 2011.

### BACKGROUND OF THE INVENTION

The present subject matter provides an improved hydration and nutrition system for athletes. More specifically, the hydration system provides an improved reservoir and delivery system that is integrated with a computerized nutritional calculator (CNC) that monitors real-time expenditure and consumption of nutrition (electrolytes and calories) and fluid under racing and/or training conditions.

There are numerous alternatives for the veloist to replenish fluids and nutrition while in motion. Each design has its own pros and cons. As the sport of bicycle, duathlon, and triathlon racing has evolved and competitive advantages become even more critical, it has become increasingly important to replace fluids and calories in a timely and appropriate manner. While we have developed technologies for measuring and monitoring real-time performance metrics on the bicycle, the same degree of sophistication has not been applied to monitoring the metabolic and homeostatic requirements of the athlete.

For the “ultra” athlete, fluid, calories, and electrolytes have to be carefully consumed—often in exaggerated quantities—on a constant basis while racing. To meet this need, many different formulations of nutritional products (juices, powdered fluid mixes, gels, energy bars, and “candies” to name a few) have been developed for the performance athlete. To most effectively meet the athlete's needs under competitive conditions, the supplies have to be readily available and presented in a manner that does not cost time, energy, and effort. It is also crucial that the athlete be aware of his status with respect to replenishment at all times since deficits lead to impaired performance and are extremely difficult to overcome during a race. There are two essential aspects to effective maintenance of fluid and caloric status: (1) efficient delivery; and (2) comprehensive information.

Accordingly, there is a need for a hydration and nutrition system, as described and claimed herein.

### SUMMARY OF THE INVENTION

In order to meet these needs and others, the hydration and nutrition system described herein provides an improved reservoir and delivery system integrated with a computerized nutritional calculator (CNC) that monitors real-time expenditure and consumption of nutrition (electrolytes and calories) and fluid. The hydration system includes two primary components: (1) the reservoir/delivery system; and (2) the CNC.

While described primarily herein with reference to triathletes and triathlons, it is understood that the hydration and nutrition system may be modified or adapted for appropriate use in numerous athletic environments, particularly those wherein the athlete exerts himself/herself over an extended time.

Reservoir and Delivery System

An incredible array of bottles and other reservoirs are currently on the market. The most prevalent is the simple squirt bottle—a staple that will inevitably survive all others because it is simple, effective, easily reseated and can be used to provide liquid under pressure. While the other products on the market may seem to be an improvement, they generally offer very few tangible benefits over the squirt bottle. Reservoir systems may offer several advantages over the simple squirt bottle including: (1) improved aerodynamics; (2) hands free tube and bite valve allows the rider to remain in a tucked, aerodynamic position while drinking; and (3) holds a large volume of fluid.

While there are definite benefits to reservoir systems, in practice it is rather difficult to suck fluid through the tubing. The passive flow system actually discourages fluid intake. There are some pressurized hydration systems on the market that attempt to address this problem, but they are either designed for backpacks or require manual pressurization by the rider “en-route.” The majority of these products pressurize the fluid reservoir via a bladder that is outside the fluid reservoir itself—a task that distracts the athlete from his performance.

The present invention provides an improved reservoir system. It offers all of the advantages of presently available reservoir systems and remedies their shortfalls. According to the teachings disclosed herein, fill bottles including one-way, spring-loaded fill valves are paired with spring-loaded receiving caps on the reservoirs. Once filled with the appropriate fluid, the fill bottles are pressurized through extension tubes that are screwed into threaded portions of the one-way valves built into the fill bottle caps. The extension tubes fit into a standard bicycle tire pump. By pressurizing the fluid in the fill bottles, the replacement fluid is injected into the reservoir when the fill bottle is properly seated in the reservoir. This greatly enhances fill rates and further results in pressurized fluid within the reservoir. Because the fluid in the reservoir itself is pressurized, the fluid is injected into the athlete's mouth, minimizing the effort required by the user. Pop-off valves in both the fill bottles and the reservoirs prevent over-pressurization. The reservoirs also include one-way vents to allow entrainment of air into the reservoir, should the pressure inside the reservoir drop too low to allow for egress of fluids into the tubing. All plastic components of the system may be silver impregnated to prevent bacterial and fungal contamination.

The reservoir and delivery system addresses the needs of the athlete who is traveling longer distances and has to keep track of replacing fluids and nutrition on their own. The system is easy to use, reduces delays during refilling and actually encourages rehydration.

For purposes of illustrating the invention, a preferred embodiment is summarized below. In this example, the reservoir system includes the following primary components: a dual-chamber reservoir; a locking fill cap, including a spring-loaded filling valve, a pressure relief valve, and a one-way air entrainment valve; an extension tube through which a pump may pressurize the reservoir; and dual-lumen straws and bite valves through which the user may receive the fluids. These elements and others are described in greater detail below.

In this example, the principal reservoir is a 48-ounce, dual chamber, pressure-sealed reservoir housed within the triangular bicycle frame. One side may be used for water and the other for an “energy drink” The reservoir fits into a cage that is bolted to the down tube of the bicycle frame.

Each side of the reservoir includes an associated removable, airtight, locking filling cap which has a built-in spring-loaded filling valve, a pressure relief valve (to prevent over-pressurization) and a spring-loaded, “failsafe,” one-way valve to allow entrainment of air, should the reservoir lose pressure (this enables the athlete to suck fluid from the reservoir if there is no pressure).

A threaded portion on top of the “failsafe” valve accommodates an extension tube (similar to a bicycle wheel valve extender) that can be used to pressurize the reservoir using a regular bicycle pump. The extension tube may be unscrewed and removed after pressurizing the reservoir.

“Straws” are inserted via threaded, airtight ports into each side of the to of the reservoir and extend inside the reservoir to the bottom of the reservoir. “Male” quick-connect valves at the top of the straws connect to corresponding “female” counterparts in dual-lumen tubing, which terminates in dual bite valves to deliver the fluid under pressure to the rider. The bite valves are designed to maintain pressure in the system.

Between the reservoir bottle and the bite valves, the tubing is extruded to become a dual-lumen tube that is ovoid in cross-section (for improved aerodynamics and reduce redundancy in the tubing). The portion of dual-lumen tubing between the dual bite valves and anchoring hardware described below includes built-in wires to hold the tubing in a bent position that allows the rider to help locate the bite valves in a convenient and ergonomic location.

In the preferred embodiments, all plastic components are silver impregnated to be bacteriostatic and fungistatic. They are further dishwasher safe and slightly opaque for aesthetic purposes.

In a preferred embodiment, the reservoir system is secured to the frame of the bicycle via specially designed anchoring hardware. The anchoring hardware performs several functions including: (1) attaches the tubing and CNC to the headset frame of the bicycle; (2) serves as an anchoring point for the wire housed within the dual-lumen tubing; and (3) houses dual flow meters that are in-line with the dual-lumen tubing and measure the fluid volumes and/or flow rates that are delivered to the athlete. This data from the flow meters is communicated wirelessly to the CNC.

As described, the reservoirs may be filled passively and then pressurized with a bicycle pump or may be filled using appropriately-sized, pressurized, fill bottles including valve systems built into the bottle's cap. The threshold for activating the pressure relief valves in the fill bottles is double the threshold for activating the pressure relief in the reservoir. The relative proportions of the thresholds are established in order to appropriately pressurize the reservoir and ensure that the filling bottle fluid is transferred to the reservoir. The fill bottles have spring loaded, “male” filling valves that connect with “female” receiving valves on the reservoir bottle. The entire filling bottle is airtight and constructed of the same plastic material as the reservoirs.

It is understood that numerous embodiments of the inventions provided herein may be employed. For example, smaller reservoir systems (30 ounce) that lack flow meters and CNCs may be used for shorter distance racing. It is contemplated that these smaller reservoir systems may differ from the 48-ounce reservoir systems in the following ways. The smaller reservoir systems may include: a single chamber and a single reservoir fill cap; a single, threaded, airtight port at the top with a single straw; a narrower cradle to house the smaller reservoir and have a single opening at the top to accommodate the port and straw; tubing including a single lumen and a single, embedded wire for positioning of the bite valve; and hardware for anchoring the tubing to the aerobar adapted for the single lumen tubing.

Reservoirs as described herein may also be designed to attach to other structures on the bicycle such as the aerobars of time trial bicycles, behind the seat or underneath the top tube. Due to limited space, reservoirs that attach to the aerobars may be designed so that the flow sensors and CNC attach to the top of the reservoir itself.

Computerized Nutritional Calculator (CNC)

Although every athlete acknowledges that optimal fluid and nutrition consumption are vital to optimal performance, most lack the sophistication to be able to achieve that goal. Without an attentive coach, athletes essentially guess how to replace fluid and nutrition deficits. The present subject matter discloses a nutritional monitor with carefully constructed, mathematical algorithms that can be applied in real-time and on an individualized basis to calculate optimized fluid and nutrition consumption during performance. By utilizing data from readily available input sources, a very sophisticated nutritional and fluid replacement guide is provided.

In a preferred embodiment, the CNC may be slightly larger than currently available cycle computers with a screen that can be easily read under direct sunlight conditions. Push buttons on either side of the screen are used for data entry and user control of other options. The CNC may include any combination of communication mechanisms, including, for example, wireless, 3G, Bluetooth and USB connectivity. The settings and parameters of the CNC may be “programmed” via a separate device, for example, from a computer through a USB cable or from a handheld device through a Bluetooth connection.

In use, the CNC keeps track of consumption and displays real-time data to the rider. In a preferred embodiment, the consumption tracked includes water, “energy drink,” and caloric intake. The athlete will be able to determine the baseline requirements for a given peak performance power output. The CNC will then use the averaged percentage of peak performance power output during the race to calculate the athlete's required rate of fluid, caloric and electrolyte replenishment rates. Alerts may be built into the programming to keep the rider from developing a deficit. The alerts may be based on one or more algorithms that take numerous factors into account, such as, ambient temperature, elevation above sea level, and heart rate to modify replenishment rates as conditions on the actual course change.

Built-in GPS allows the CNC to track consumption of fluids and calories over each traveled route. This data can then be overlaid on changes in topography, speed, distance traveled and other performance metrics that can either be directly measured or extrapolated from the data that is measured. Using algorithms based on data collected directly from studying other athletes, the CNC can extrapolate calorie expenditure using the user's weight and distance traveled. This data can then be used to match calorie expenditure with consumption. Data can also be uploaded into the computer program to create profiles for the user. These profiles can then be displayed in graphic form and also be used to improve accuracy with estimating future requirements for each specific athlete.

Computer management software embodied in a separate electronic device (e.g., computer, mobile device, etc.) may interface with the CNC. The software may allow the athlete to download data from the CNC to “track” consumption during training exercises to determine optimal fluid and nutritional requirements. The interfaced software may also allow the athlete to customize settings on the CNC for use while racing. The software may also include and/or be updated to include caloric and electrolyte values for energy drinks, gels, bars, foodstuffs, etc. These values may then be uploaded into the CNC and used in analyzing the user's consumption. The CNC buttons may be “assigned” to specific foodstuffs using the computer interface. Then, when riding, pressing the buttons tells the CNC when and which of those foodstuffs are consumed.

The CNC may include a built-in thermometer that uses ambient temperature to modify fluid requirements according to established algorithms. The ambient temperature may further be used by the CNC to estimate caloric expenditure.

Wired and/or wireless connectivity enables the CNC to synchronize and otherwise receive data from cyclocomputers and/or other sensors such as speed, cadence, heart rate and power output. This data can then be used together with GPS data and temperature data for determination of caloric expenditure based on athlete weight and body surface area. The CNC may be adapted to interface with any wireless protocol, including, for example, ANT+, GPRS, CDMA, EV-DO, EDGE, 3GSM, DECT, IS-136/TDMA, iDen and LTE, to name a few of the wireless technologies that are currently in use. Accordingly, in some embodiments, the CNC may incorporate all of the functions of a traditional cyclocomputer and display performance metrics like speed, cadence, heart rate and power output.

In a preferred embodiment, the CNC can be removed from the handlebar/aerobar mounting hardware and attach to an identical mount on a wristband to continue tracking caloric consumption and fluid replacement, for example, during a run phase of a triathlon. During the run phase, consumption data may be entered manually using pre-programmed buttons. In such instances, once removed from the vicinity of the flow sensor controller, the CNC automatically assumes that the run phase has been initiated and adapts the monitoring functions appropriately.

The CNC display is adapted to show data in an easy to read format. Fluid and calories may be displayed as “per hour” rates as well as (or alternatively as) totals consumed. Arrows pointing up, down and horizontal may be used to indicate surplus, deficit, and “on target,” respectively, for each value. These arrows blink when deficits or surpluses exceed pre-established confidence limits. In addition to hart rate, speed, pedaling cadence, and power output, lapsed time, miles traveled, ambient temperature, fluid and caloric consumption rates are also displayed. Icons adjacent to “input” buttons can be changed using a computer or handheld device to aid the user in correctly entering foods and fluids consumed outside of the flow meter input. Such items include “energy” gels, bars and candies, fruit, sandwiches, and other calorie rich foods. Performance metrics can also be displayed in the usual and customary manner of typical cyclocomputers.

It is know that by using performance monitors like computrainers and power meters (powertaps), athletes can determine a standard measurement of their combined strength and endurance. This physiologic metric is referred to as the Functional Threshold Power (FTP) and usually expressed in averaged watts an athlete can sustain for a period of 1 hour.

In a presently contemplated example of the hydration and nutrition system, FTP may be used as a primary independent variable in an equation used to calculate fluid and nutritional requirements of the user. Ambient temperature, elevation above sea level, age, sex, heart rate, and calculated deficits from the swim phase of the race, may be modifiers to the calculated replacement rates. The measured or extrapolated percentage of FTP at which the athlete is performing may automatically reflect the topographical and wind-adjusted conditions of the course and these variables will not be necessary for calculating replacement quantities.

The initial fluid and caloric replacement rates may be determined using variables like age, sex, height, weight (BMI), calculated body surface area, resting heart rate, and peak heart rate. “Baseline” replacement values may be determined by a marriage of existing normograms and measured normograms—i.e., the athlete will input these values prior to using the system for the first time. These values would then be used to program the CNC for replacement quantities at 100% of FTP.

After uploading the data collected from each ride into an associated device (e.g., a tablet or computer), the athlete can then analyze the performance metrics measured during the ride and evaluate the efficacy of the fluid and nutritional replacement. At this time, the athlete can modify the baseline replacement rate up or down at points along the route based on observed responses to the replacement rate. The system thus “learns” from the athlete and adjusts accordingly for the next ride or race.

Of course, it is contemplated that there may be a more useful variable than FTP or that multiple variables may be used to improve the algorithmic functions of the present systems and methods. The examples provided herein based on FTP are for illustrative purposes of a presently preferred embodiment, but are not a limitation on the application of the systems and methods.

In one example, a hydration and nutrition system for monitoring consumption of fluid by a user includes: a reservoir adapted to hold a quantity of fluid; a tube adapted to convey the fluid from the reservoir to the user for consumption; a flow meter adapted to measure and collect data regarding the flow of fluid from the reservoir through the tube and communicate the data regarding the flow of fluid to a computerized nutritional calculator, wherein the computerized nutritional calculator is adapted to calculate a deficit or surplus of fluid consumed as compared to a consumption plan.

In a specific example, the hydration and nutrition system may include a reservoir adapted to hold two quantities of fluid in separate reservoirs and the tube adapted to convey the fluid from the reservoir to the user for consumption may be a dual-lumen tube. Each lumen in the tube may be associated with one of the two reservoirs. The hydration and nutrition system may further include a tube extending into each of the reservoirs, wherein each of the tubes extending into the reservoirs connects to a respective lumen of the dual-lumen tube. Each of the tubes extending into each of the reservoirs may connect to the respective lumen of the dual-lumen tube through a quick connect. Each of the lumens in the dual-lumen tube may include a bite valve and each bite valve may be connected to the dual-lumen tube via a swivel quick connect.

The reservoir may include a fill cap including a pressure relief valve and a one-way air entrainment valve. The hydration and nutrition system may further include a fill bottle adapted to mate with the fill cap, wherein the fill bottle includes a pressure relief valve and a one-way air entrainment valve.

The computerized nutritional calculator may calculate volumes of fluid consumed and/or caloric intake for at least one fluid. The computerized nutritional calculator may calculate a flow rate of fluid consumed compared to a flow rate in the consumption plan. The consumption plan may be pre-determined and/or modified by conditions measured over a period of time defining the consumption plan. The conditions measured over a period of time defining the consumption plan may include location and temperature data collected by a location sensor and a temperature sensor associated with the computerized nutritional calculator. The conditions measured over a period of time defining the consumption plan may further, or alternatively, include data received from bicycle performance sensors.

The computerized nutritional calculator may switch from a first consumption plan to a second consumption plan when the computerized nutritional calculator is no longer receiving data from the flow meter.

An example of a method of monitoring consumption of fluid by a user during an athletic event includes the steps of: providing a hydration and nutrition system for monitoring consumption of fluid by a user comprising: a reservoir adapted to hold a quantity of fluid; a tube adapted to convey the fluid from the reservoir to the user for consumption; a flow meter adapted to measure and collect data regarding the flow of fluid from the reservoir through the tube and communicate the data regarding the flow of fluid to a computerized nutritional calculator; providing a consumption plan defining desired replacement of fluid deficits; and calculating a deficit or surplus of fluid consumed as compared to the consumption plan. The consumption plan may further define desired replacement of nutritional deficits and the calculation of the deficit may include monitoring of real-time conditions throughout a period of time defining the consumption plan.

The systems and methods taught herein provide efficient and effective solutions for an improved hydration and nutrition system for athletes.

An advantage of the hydration and nutrition system provided herein is in the improved reservoir system for supplying fluids to the cyclist.

Another advantage of the hydration and nutrition system provided herein is in the automated monitoring and analysis of the user's consumption of fluids and other nutritional elements.

A further advantage of the hydration and nutrition system provided herein is in providing pressurized fluid delivery. Pressurizing fluid encourages athletes to drink because it requires less energy than sucking. It is the cornerstone of the design of the reservoirs. It also prevents backflow of fluid in the tubing, which affects flow sensor accuracy.

Another advantage of the hydration and nutrition system provided herein is in the techniques provided for pressurizing the reservoir. The use of pressurized fill bottles and the specially designed fill caps provides an elegant solution for pressurization because it negates the need for a pressurization device attached to the reservoir, reduces the time needed for filling the reservoir (very important in race conditions), prevents spillage, and saves weight because there is no additional piece of equipment needed to maintain pressurization (weight reduction is critical—it costs about $750 per pound to make a bicycle lighter). Energy drinks contain a lot of salt and spillage through “leaky” fill caps on currently available reservoir systems damages the bearings of the wheel axels and the crank—all expensive replacements.

Yet another advantage of the hydration and nutrition system provided herein is the use of straws screwed into threaded ports in the reservoir for draining the most gravity dependent portion of the reservoir and preserving pressure in the reservoir. Current systems typically use rubber gaskets that leak. The quick-connects provided herein make it much easier to attach and detach the tubing.

Another advantage of the hydration and nutrition system provided herein is the use of dual-lumen tubing for the drinking straw. The double-lumen tubing saves weight and is aerodynamic.

Still another advantage of the hydration and nutrition system provided herein is the use of flow sensors that can be replaced if damaged or infected with bacteria or fungi. For example, the use of infrared sensors with disposable components may be easier to clean.

A further advantage of the hydration and nutrition system provided herein is the integration of a cradle that uses a clasp mechanism to facilitate insertion and removal of the reservoir. The clasp design is unique and an improvement over existing cradle/reservoir fastening methods.

Yet another advantage of the hydration and nutrition system provided herein is in its adaptability. Many of the unique properties of this reservoir system can be applied to a reservoir placed anywhere on the bicycle. This allows the athlete to use the system with their preference of reservoir position.

Still another advantage of the hydration and nutrition system provided herein is the intelligent monitoring and guidance with respect to hydration and nutrition will help amateurs and competitive athletes to learn how best to manage their intake and optimize their performance. Failure to maintain an appropriate replacement rate affects nearly every professional athlete at some point in his or her career.

Additional objects, advantages and novel features of the present subject matter will be set forth in the following description and will be apparent to those having ordinary skill in the art in light of the disclosure provided herein. The objects and advantages of the invention may be realized through the disclosed embodiments, including those particularly identified in the appended claims.

### BRIEF DESCRIPTION OF THE DRAWINGS

The drawings depict one or more implementations of the present subject matter by way of example, not by way of limitation. In the figures, the reference numbers refer to the same or similar elements across the various drawings.

FIG. 1 is a perspective view of an example of a hydration and nutrition system adapted for use on a bicycle.

FIG. 2 is a side view of an example of a reservoir.

FIG. 3 is a front view of the reservoir shown in FIG. 2.

FIG. 4 is a top view of the reservoir shown in FIG. 2.

FIG. 5 is a cross-sectional view of the reservoir shown in FIG. 2.

FIG. 6 is a top view of a fill bottle.

FIG. 7 is a side view of the fill bottle shown in FIG. 6.

FIG. 8 is a front view of the fill bottle shown in FIG. 6.

FIG. 9 is an exploded assembly view of a reservoir, fill bottle, mounting assembly, and various associated elements.

FIG. 10 is a cross-sectional view of the interface between the reservoir and fill bottle shown in FIG. 9.

FIG. 11 is a cross-sectional view of the interface between the reservoir and an extension tube.

FIG. 12 is a front view of the hydration and nutrition system particularly illustrating bite valves and quick connects.

FIG. 13 is a cross-sectional view of the dual-lumen tubing connecting the reservoir to the bite valves.

FIG. 14 is a perspective view of the hydration and nutrition system shown in FIG. 12.

FIG. 15 is a front side view of a computerized nutritional calculator.

FIG. 16 is a perspective view of the computerized nutritional calculator adapted for use on a wristband.

### DETAILED DESCRIPTION OF THE INVENTION

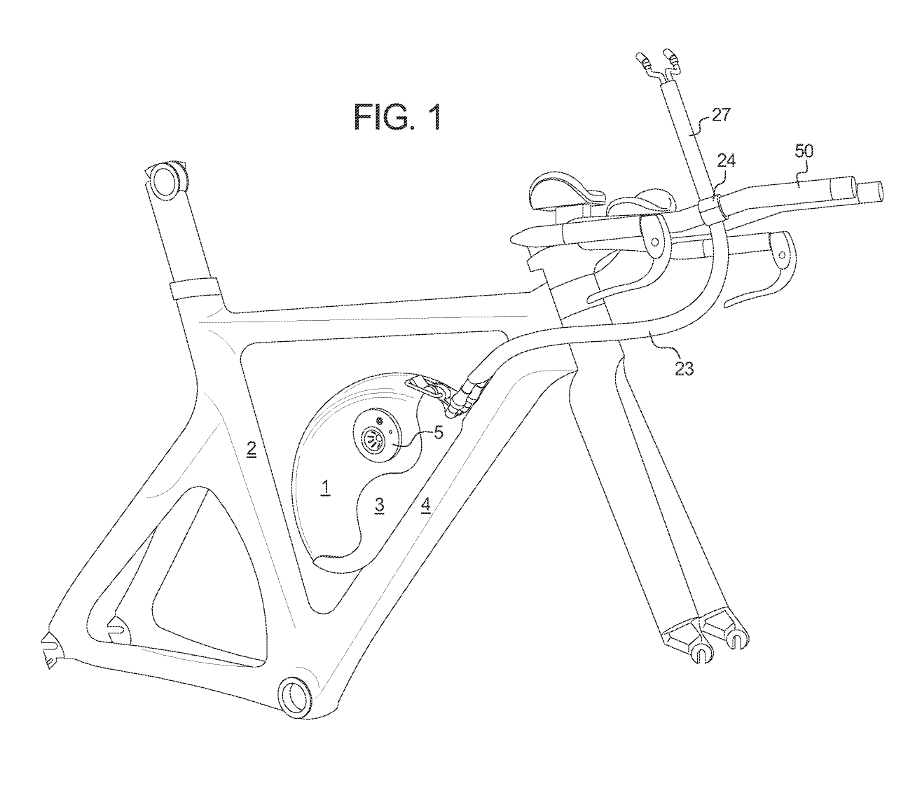

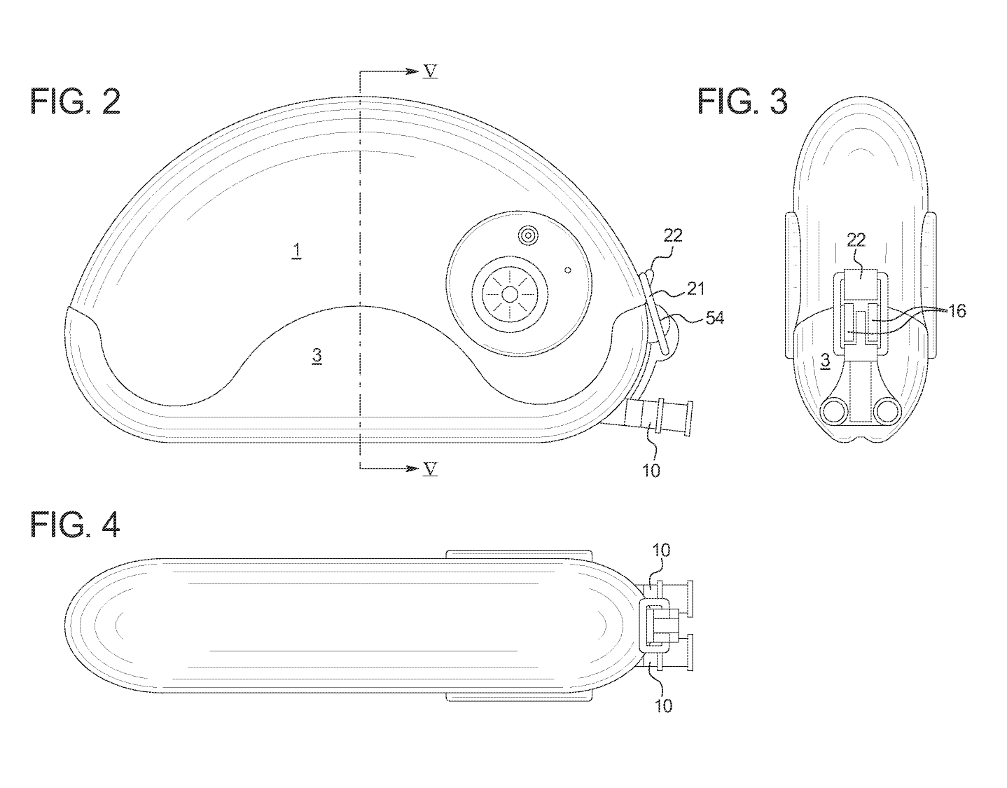

FIG. 1 illustrates a preferred embodiment of a hydration and nutrition system according to the present invention. As shown in FIG. 1, the system includes a reservoir system including a reservoir 1 that attaches to a bicycle frame 2. In the example shown in FIG. 1, the reservoir 1 locks into a cage 3 which mounts to a down tube 4 of the bicycle frame 2. However, as described further herein, the location of the reservoir 1 on the down tube 4 is merely one contemplated embodiment and the location of the reservoir 1 may vary as will be understood by one skilled in the art based on the disclosure provided herein. FIGS. 2-4 further illustrate the elements of the reservoir 1 and the cage 3 shown in FIG. 1.

In the example shown in FIGS. 1-4, the cage 3 is constructed of a durable plastic (such as polyethylene terephthalate) and is engineered to be more robust than the reservoir 1. Carbon fiber is an alternative, though more expensive, material from which to construct the cage 3. In the example shown, two screws 19 placed through a slot 15 in the base of the cage 3 allow for positioning of the cage 3 along the down tube 4 of the bicycle frame 2 and anchor it to two threaded holes 20 in the down tube 4 that are usually used to anchor bottle cages. The cage 3 is robust enough to support a full reservoir 1 through the rigors of rough road conditions and shear forces exerted during the filling process.

The design of the cage 3 may be integrated with the design of the reservoir 1 in such a way as to be aerodynamic and aesthetically pleasing. For example, in one contemplated version, a knob on the bottom of the cradle 3 fits into a notch in the bottom of the reservoir 1 and serves to anchor the bottom of the reservoir 1. In the example shown, there is an extruded knob 22 along the top of the reservoir 1 to which a clasp 21 at the top of the cradle 3 secures the reservoir 1 into the cradle 3 in a manner similar to a ski boot binding or a jar closure. Further in the example shown, the top part of the cradle 3 is divided (or provides a slot) to allow a pair of straws 10 to pass through and be positioned properly within the reservoir as described further herein. Accordingly, as shown in FIG. 3, raised portions 16 in the two divided sides of the cradle 3 are clamped together by the clasp 21 at the top of the cradle 3 to further secure the reservoir 1 into the cage 3.

The reservoir 1 may be made of a lightweight, yet durable plastic (such as polyethylene terephthalate) that is slightly opaque in color for aesthetic purposes. A gloss exterior finish may be used for aesthetic purposes as well. Because of polyethylene terephthalate's durability, the thickness of the plastic can be significantly thinner than currently available PVC reservoir systems.

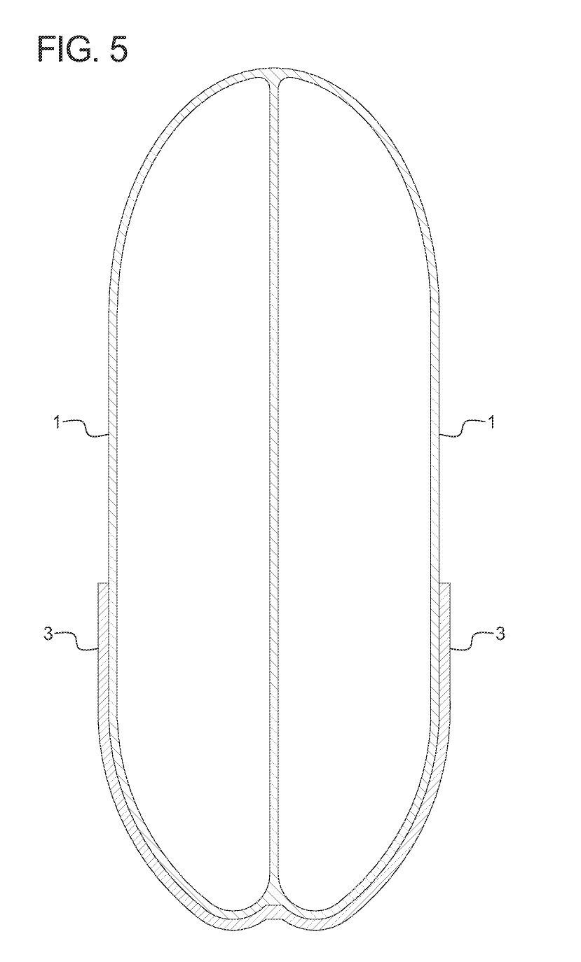

Larger versions of the reservoir 1 (e.g., 48 ounce version or 30 ounce version) may be formed from two “mirror image” halves that are welded together to form an aerodynamic whole, as shown in FIG. 5. Each of the halves may be used to store a different fluid, as is described further herein. Accordingly, each half may be considered its own distinct reservoir 1. It is also contemplated that single-fluid reservoirs 1 may be employed, particularly in smaller sized reservoirs 1.

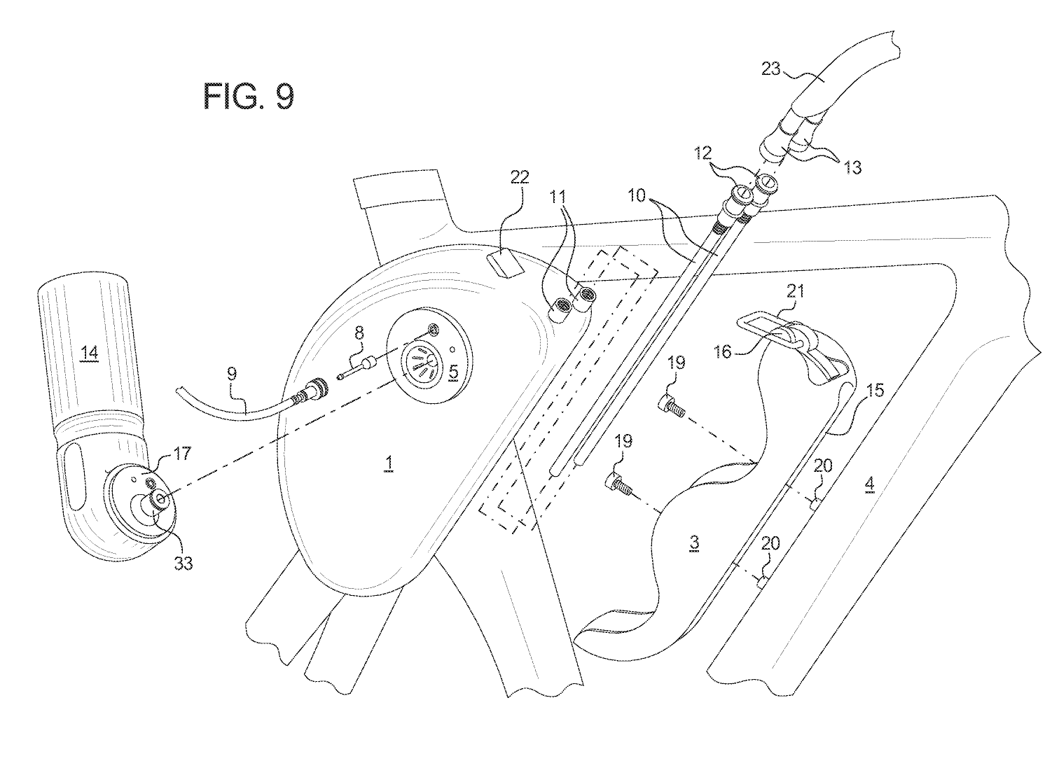

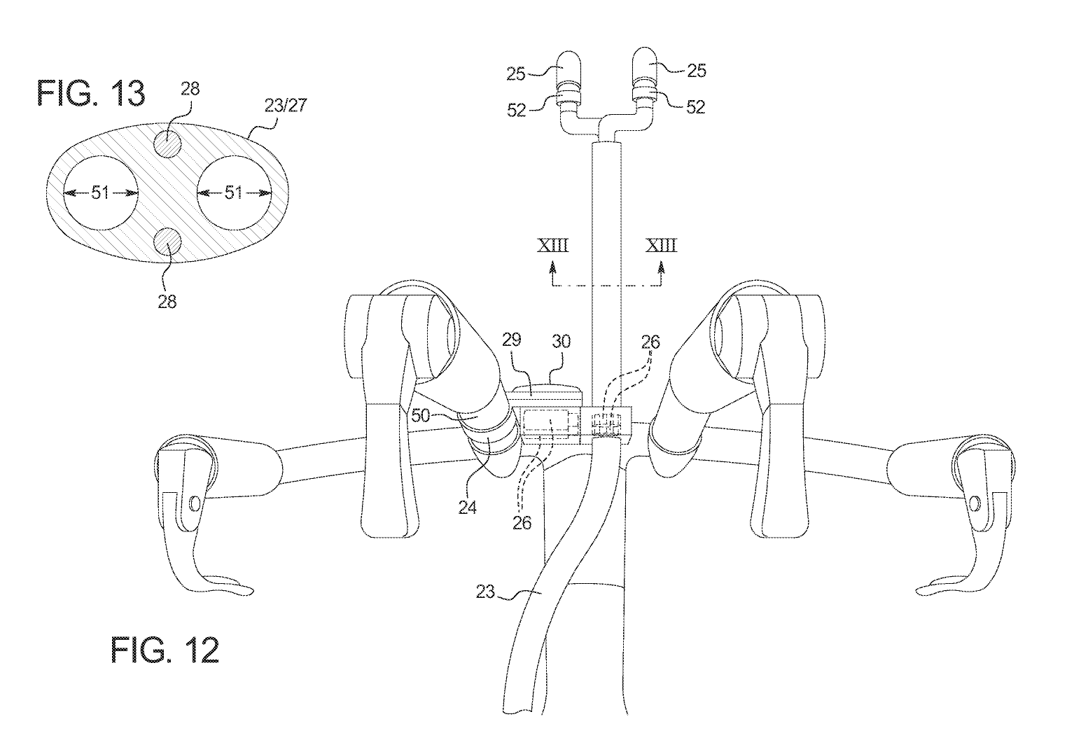

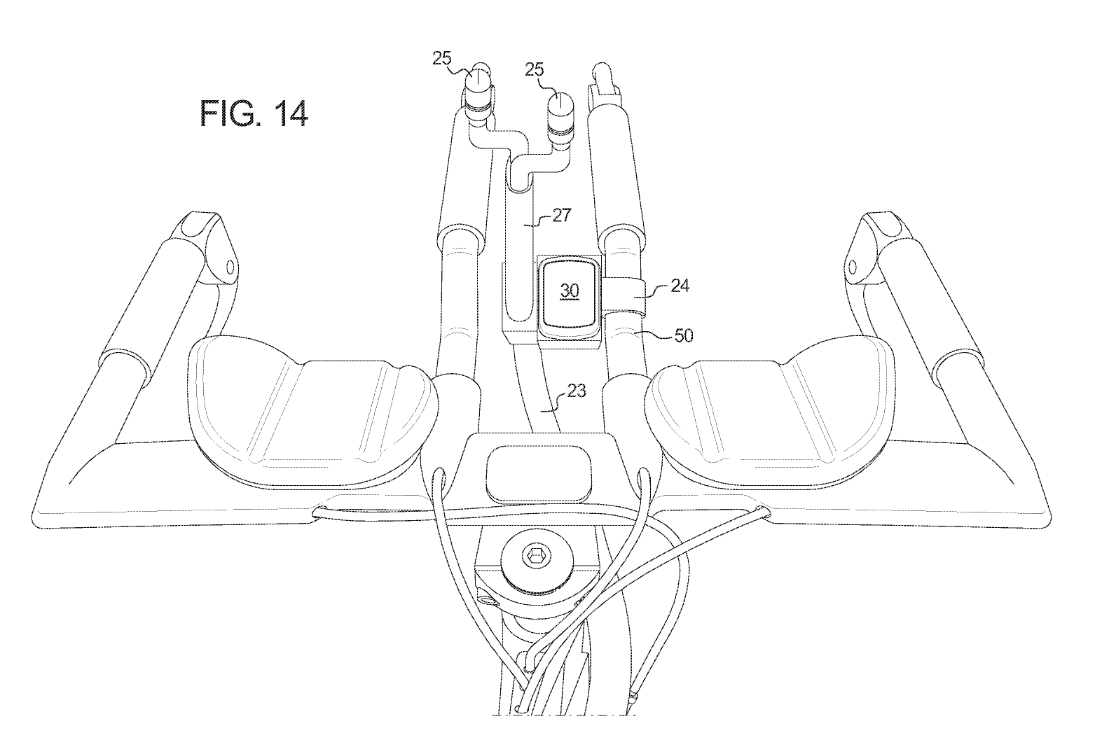

As shown in FIGS. 9-11, a threaded hole near the top of each side of the reservoir 1 accepts a specially designed filling cap 5. At the top end of each side of the reservoir 1 is a threaded port 11 into which a threaded tube/straw 10 is screwed in an airtight manner. Male quick connects 12 at the top of these tubes/straws 10 “mate” with a female quick connects 13 on the bottom of a dual-lumen tube 23 that runs up to a CNC 30 (FIGS. 12 and 14-16). The straws 10 and fill caps 5 can be removed for cleaning. Each of the reservoirs 1, caps 5, straws 10, and tubing 23 may be formed from polyethylene terephthalate and may be impregnated with silver in order to be bacteristatic and fungistatic. The various elements may also be dishwasher-safe.

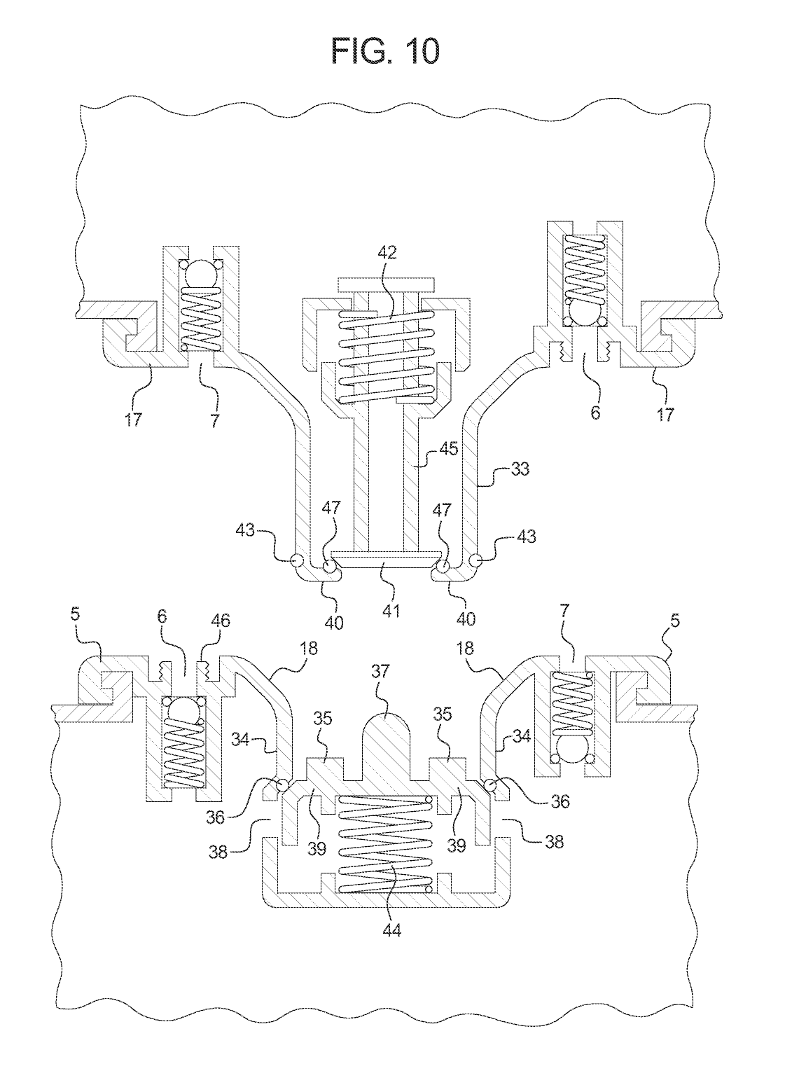

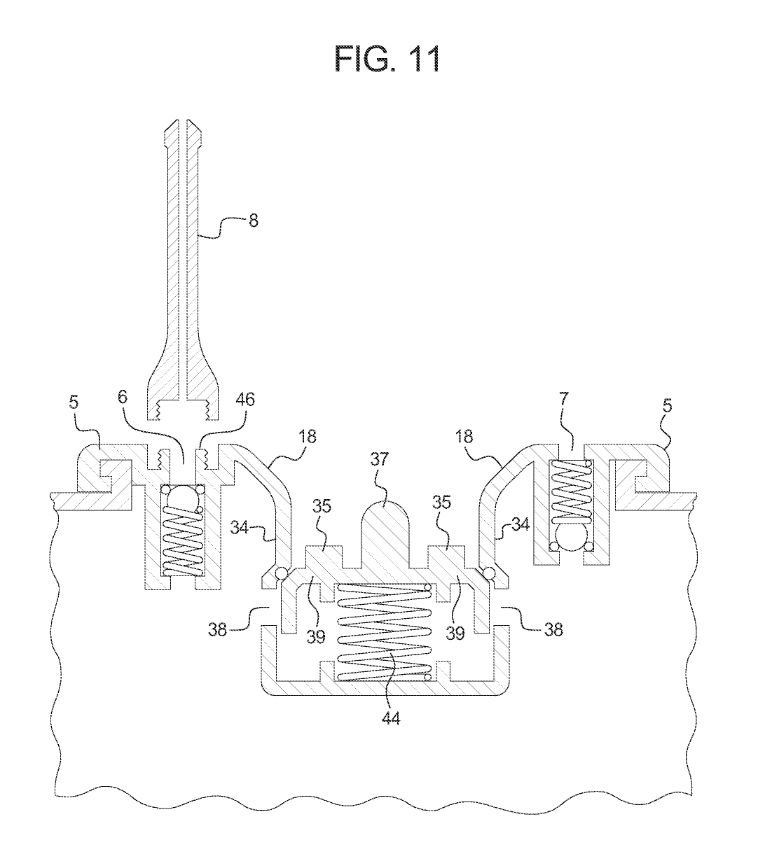

In the examples shown in FIGS. 9-11, the reservoir fill cap 5 screws into the reservoir 1 and is pressure sealed. The center of the reservoir fill cap 5 is chamfered 18 to direct a fill bottle nozzle 33 into the center of the reservoir fill cap 5. In the center of the reservoir fill cap 5, there is a cylinder 34. The sides of the cylinder 34 are perforated 38 to allow filling of the reservoir 1. As shown in FIGS. 10 and 11, a spring 44 in the “base” of the cylinder 34 pushes a sealing disc 39 against an O-ring 36 in the cylinder 34 to create a pressure seal. When the fill bottle nozzle 33 is engaged in the reservoir fill cap 5, the fill bottle nozzle 33 pushes the sealing disc 39 in the reservoir fill cap 5 inwards and allows pressurized fluid to enter the reservoir 1 through the perforations 38 in the side of the cylinder 34. The reservoir sealing disc 39 is molded to include an elevated center 37 that pushes a sealing disc 41 in the fill bottle nozzle 33 inwards, allowing fluid to enter the reservoir 1. Circumferentially molded vanes 35 in the reservoir fill cap sealing disc 39 prevent the front end 40 of the fill bottle nozzle 33 from sealing against the reservoir cap sealing disc 39 and directs fluid into the reservoir 1 in an even, 360 degree, manner. An O-ring 43 in the wall of the fill bottle nozzle 33 helps to maintain pressure integrity during filling. When the fill bottle 14 is removed, the reservoir fill cap sealing disc 39 returns to abut against the O-ring 36 in the cylinder 34 at the center of the reservoir fill cap 5.

As shown, two valves housed within the reservoir fill cap 5. The first is a pressure relief valve 7 that limits the maximum pressure that can build up inside the reservoir 1. The other is a one-way valve 6 that will allow for entrainment of air into the reservoir 1, should all of the pressure in the reservoir 1 be lost. The one-way valve 6 acts as a failsafe that will allow the athlete to suck fluid out of the reservoir 1. There are several different ways in which each of the valves can be designed—the function is more relevant than the form. A threaded portion 46 on top of the failsafe valve 6 will accommodate an extension tube 8 (similar to a bicycle wheel valve extender) that can be used to pressurize the reservoir 1 using a standard bicycle pump 9, as shown in FIGS. 9 and 11.

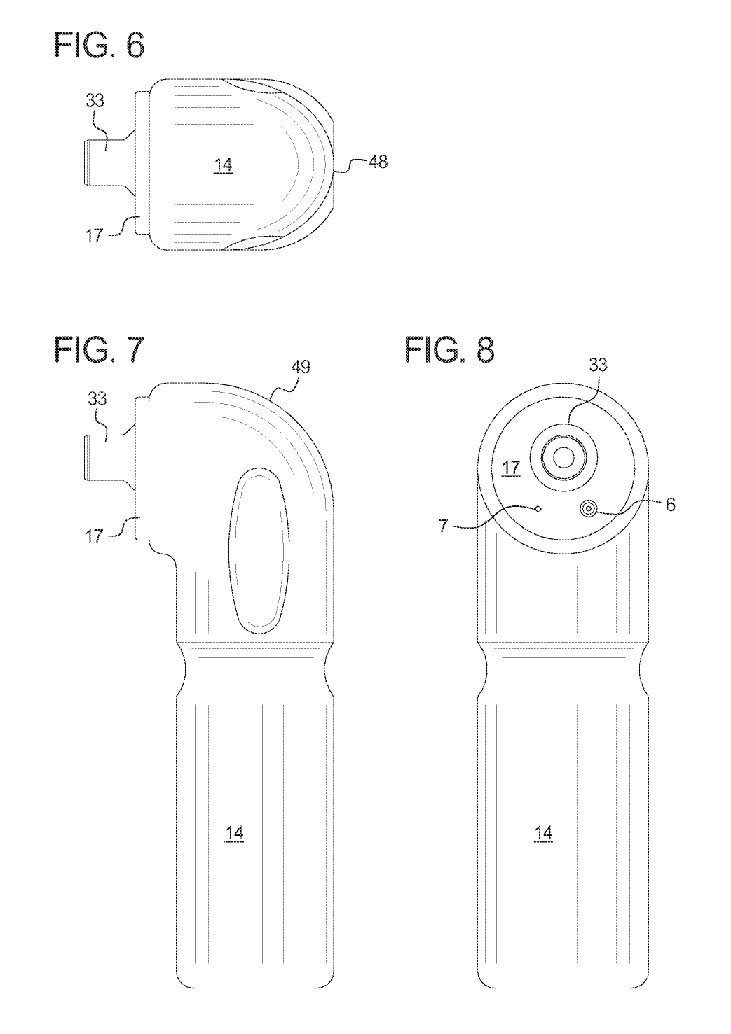

An example of a fill bottle 14 is shown in FIGS. 6-10. The fill bottle 14 is designed to be pressurized once full of water or energy drink. The pressure in the fill bottle 14 is used to push fluid into the reservoir 1, which speeds filling time and pressurizes the fluid in the reservoir 1. In a preferred embodiment, the pressure in the fill bottle 14 should be double that of the desired pressure in the reservoir 1. In a preferred embodiment, a side of the fill bottle 14 is flat to enable it to be placed securely on a flat surface for filling.

In the examples shown, the reservoir fill caps 5 and the fill bottle caps 17 have identical one-way valves 6 that are used to pressurize their respective containers using a threaded tube 8 and a standard bicycle pump 9. In use, once the correct pressure in the reservoir 1 has been reached, air will be heard exiting from the pressure relief valve 7 in the reservoir cap 5 and/or fill bottle cap 17. Use of carbon dioxide canisters to pressurize the containers is specifically discouraged. The fill bottle cap 17 includes a pressure relief valve 7 that is set for double the desired pressure in the reservoir 1.

The fill bottle 14 is designed to fill the reservoir 1 while being held “upside-down” and has a semi-circular top end 49 to reduce the residual volume in the fill bottle 14 after filling is completed. The fill bottle cap 17 is located on the side of the fill bottle 14 to ensure maximal transfer of fluid to the reservoir 1. The side of the fill bottle 14 opposite that of the fill bottle cap 17 is flattened 48 so that the fill bottle 14 can be placed on a flat surface during pressurization.

The fill bottle cap 17 has many of the same features as the reservoir fill cap 5, but is designed as a male filling counterpart to the female reservoir fill cap 5. A spring-loaded 42, sealing disc 41 is pushed into the fill bottle 14 when engaged with the raised portion 37 of the sealing disc 39 in the reservoir fill cap 5. Pressurized fluid exits the fill bottle 14 around the sealing disc 41 and enters the reservoir 1 through the opened space around the sealing disc 41 in the reservoir fill cap 5. Vanes 35 on the face of the reservoir sealing cap 39 prevent the front of the nozzle 40 of the filling cap 17 from sealing against the reservoir sealing cap 39 and ensure even filling through the reservoir filling cap 5. An O-ring 43 in the sidewall of the fill bottle cap nozzle 33 maintains the pressure seal during filling. When the fill bottle cap nozzle 33 is withdrawn from the reservoir fill cap 5, the fill bottle sealing disc 41 seals against an O-ring 47 on the inside of the front of the fill bottle nozzle 40.

Turning now to FIGS. 12 and 13, once filled, fluid in the reservoir 1 may be conveyed to the rider through dual-lumen tubing 23 and 27. Because the fluid in the reservoir 1 is pressurized, ¼″ inside diameter tubing 51 may be adequate. The dual-lumen tubing 23 and 27 may be made of pliable plastic such as ether-based polyurethane and be as lightweight as possible by having a thin wall. In the example shown, the proximal end of the tubing 23 includes female quick connects 13 that couple with male, counterpart quick connects 12 that are at the top of straws 10 on each side of the top of the reservoir 1. As described above, molded, threaded, ports 11 on each side of the top of the reservoir 1 accept straws 10 with threaded cuffs and male quick connects 12. The straws 10 extend inside the reservoir 1 to the bottom each half of the reservoir 1 in order to be able to fully empty each side of the reservoir 1. All components, including the fill caps 5 and 17 and straws 10, can be removed from the reservoir 1 in order to clean all components thoroughly and all components may be dishwasher safe.

In the preferred embodiment shown, the dual-lumen tubing 23 and 27 is ovoid in cross-section for improved aerodynamics. This dual-lumen tubing 23 attaches to flow sensors 26 in the base of the CNC/flow sensor mount 29. The proximal portion of a second section of dual-lumen tubing 27 attaches to the upper portion of the flow sensors 26. This segment of dual-lumen tubing 27 may include parallel wires 28 embedded in the dual-lumen tubing 23 and 27 to allow the athlete to bend the dual-lumen tubing 23 and 27 to an appropriate location for drinking. Dual bite valves 25 attach to swivel quick connects 52 at the “rider” end of the dual-lumen tubing 27 in order to separate the bite valves 25 for ease of use.

As shown in FIG. 12, dual flow meters 26 are housed within the mount 29 for the CNC 30 (one for each side of the reservoir 1). Numerous options for flow sensor technology exist, including turbines/paddlewheels, infrared and ultrasonic, etc. It is presently believed that the most suitable flow meter technology would be to use infrared sensors with the component that is in-line with the dual-lumen tubing 23 and 27 being detachable and potentially disposable. If not disposable, it may be advantageous if the flow meters 26 may be removed from the CNC mount 29 for cleaning. For optimal performance, the flow meters 26 should be lightweight and not obstruct fluid flow. In the example shown, the flow meters 26 are controlled by a microchip that communicates either wired or wirelessly with the CNC 30. Data from the flow meters 26 may be used to calculate volumes of fluid consumed, as well as caloric intake from energy drinks, as described further herein.

In the example shown, each bite valve 25 connects to the dual-lumen tubing 23 and 27 via a swivel quick connect 52, which allows for adjacent positioning of the bite valves 25. There are many designs for bite valves 25 that are currently available. The bite valves 25 are needed to maintain pressure within the system, so it is necessary for the athlete to apply a robust amount of “bite pressure” to open each bite valve 25. The pressure in the dual-lumen tubing 23 and 27 compensates for the bite force energy used by squirting fluid into the athlete's mouth—a passive filling experience for the athlete. The athlete is able to control the rate of filling by adjusting the bite pressure and intermittently sealing the bite valve 25 with the tongue during drinking.

A CNC mount 29 and handlebar/aerobar mounting hardware 24 are shown in FIG. 12. The CNC mount 29 and handlebar/aerobar mounting hardware 24 may be constructed of durable plastic (such as polyethylene terephthalate and/or carbon fiber) and provide several functions. First, they anchor the CNC 30 to either the aerobar 50 or to the handle bar itself via a clamp mechanism 24. Additional hardware allows for multi-directional clamping capability (attach to either side aerobar 50 or the handlebar if the aerobar 50 is an unsuitable location) as well as facilitating final positioning of the CNC 30. The clamp itself 24 may be adjustable to fit all relevant diameters of handlebar and aerobar tubing. Second, the CNC mount 29 and handlebar/aerobar mounting hardware 24 may house a microchip controller, battery cage, sensors, etc. for the dual flow meters 26. The CNC mount 29 may provide a cradle adaptor for mounting CNC 30. In addition, as shown in FIG. 16, a wristband 32 may be provided that has an identical cradle adaptor for the CNC 30.

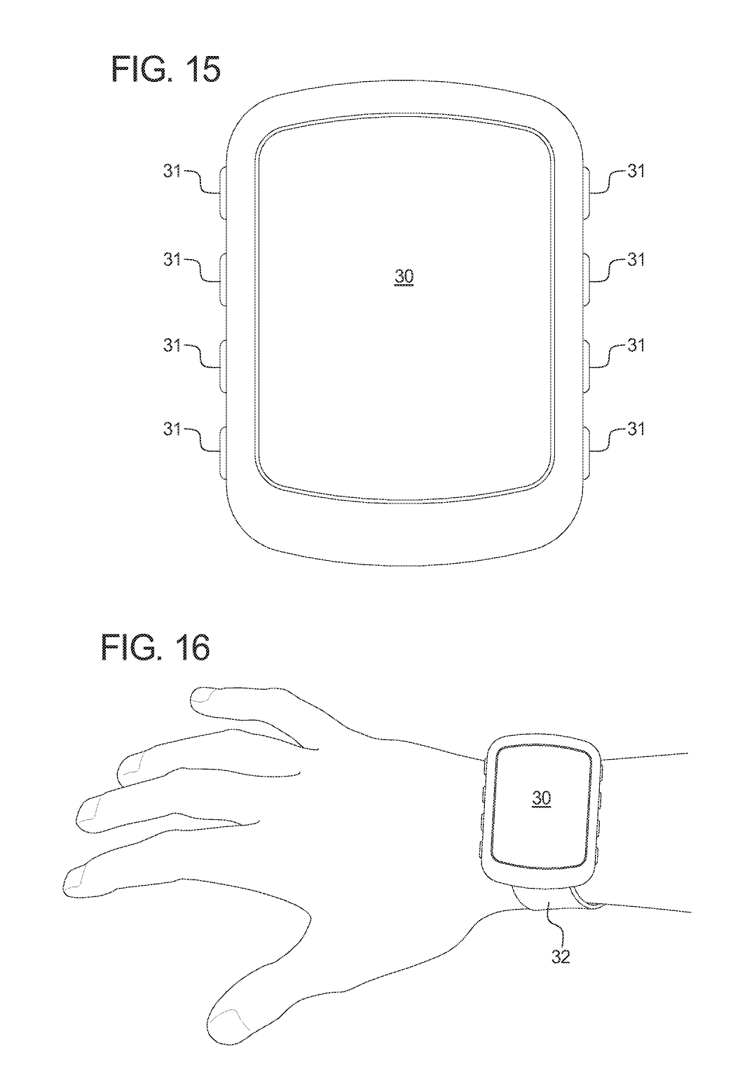

FIG. 15 shows an example of a CNC 30. As described herein, the primary function of the CNC 30 is to display measured data and express the data in the context of expected targets for nutritional consumption. Expected targets can either be pre-determined by parameters that are set by the user or modified by changes in conditions during the race. The display of the CNC 30 thus functions as a personal coach, guiding the athlete to achieve optimal fluid, calories and electrolytes during the training session or race. A memory chip in the CNC 30 may be used to store data from each session that can be uploaded to an associated computer and/or mobile device, which may further be used to program changes in the CNC 30 based on input from the user.

The CNC 30 may be adapted such that it has the ability to gather data from multiple sources. For example, the primary source of input may be through wireless communication wireless, but the CNC 30 may also be adapted to gather data internally through a built-in GPS chip and temperature sensor. Further, a USB port may provide a conduit to a computer interface and function as a charger for the CNC battery. The CNC 30 may also have Bluetooth and WiFi capability to interface with both computers and/or mobile devices. In the example shown in FIG. 15, the rider can also use a series of four buttons 31 on either side of the CNC 30 to enter data and control the function of the CNC 30.

Firmware in the CNC 30 allows it to pair with various wired and/or wireless sensors on the bicycle to monitor performance metrics like speed, pedaling cadence, power output, and rider heart rate. The CNC 30 may further be paired with the dual flow sensors 26 housed in the CNC mount 29. Personal data like age, weight, height, and sex may be entered using the buttons 31 on the CNC 30 or using an associated computer and/or mobile device. All data from performance sensors on the bicycle (e.g., speed, cadence, power output, and heart rate) may be used by the CNC 30 to determine calorie expenditure and estimate fluid replenishment needs. Although the firmware of the CNC 30 may include pre-programmed default settings for fluid, electrolyte, and calorie expenditure/replenishment needs, these settings can be refined by the user using the CNC 30 or another associated computer and/or handheld device. The temperature sensor in or associated with the CNC may be used, for example, to increase estimates of fluid requirements if ambient temperature is above expected levels.

The CNC 30 and/or the software of an associated computer and/or mobile device may interface with a website through which the user can download various data for the CNC 30, including: firmware or software upgrades for the CNC 30; nutritional information about caloric and electrolyte content of foodstuffs; suggested, pre-programmed profiles based on the user's physical biometrics, etc. The pre-programmed profiles may include recommended mixes of fluids, gels, and solid foodstuffs to be consumed during a race. In certain contemplated embodiments, registered users may also be able to get individual coaching from sports nutrition experts via an associated website. Uploading the data from the CNC 30 from each training ride and/or run will allow the user to refine the accuracy of the CNC 30 by identifying times during the ride/run where performance was optimal.

Using an associated computer interface and/or mobile device, the user can “tell” the CNC 30 which side of the reservoir 1 has water and which side has energy drink. By identifying the specific types of fluids, the CNC 30 may know the electrolyte and caloric value of the liquid. The user can also designate buttons 31 on the side of the CNC 30 to represent foods to be consumed. Icons representing those foods may appear on the screen adjacent to those buttons 31. Caloric data for those foods may be downloaded via the website and transferred to the CNC 30. The user may press the corresponding button 31 whenever a particular food item is consumed.

Ultimately, the role of the CNC 30 is to guide the user to consume fluid, electrolytes, and calories in a consistent manner so as to replace losses effectively. The human stomach can only handle approximately 1.2 liters of fluid per hour and adding foodstuffs attenuates that process. Maintaining a steady rate of consumption of a mixture of all required foods is essential to a successful strategy. In addition to actual amounts consumed, the CNC 30 may display overall caloric consumption and use arrows or other icons or indicators to indicate whether the user is behind, ahead, or on-track with the target consumption of fluids and all foodstuffs. Amounts consumed can be displayed as rates or actual amounts. In addition, the CNC may be adapted to display performance metrics, obviating the need for a second cyclocomputer.

At the end of the cycle portion of a duathlon or triathlon, the CNC 30 can be detached from the bicycle mount 29 and attached to a wristband 32, as shown in FIG. 16. As liquids and foodstuffs are consumed, the data may be entered into the CNC 30 by pressing the appropriate buttons 31. The CNC 30 may be adapted to automatically switch to “wristband mode” and continue to coach the user during the run as soon as the flow sensors 26 are no longer detected. Running has different caloric requirements than cycling. Accordingly, a separate profile for the run phase may also be programmed into the CNC 30. As is the case with the bicycle program, the run program can also be refined by downloading the data and making adjustments on an associated computer and/or mobile device.

Based on the disclosure and teachings provided herein, it is understood that those skilled in the art will recognize that the location of the reservoir 1 on the down tube 4 is a preferred embodiment, but a reservoir 1 may be located on other positions on the bicycle frame 2—for example between the aerobars 50, under the top tube (connects the handlebar stem to the saddle area), behind the saddle, etc.

For example, while FIGS. 1-4 illustrate a reservoir 1 that is positioned on the downtube 4, it is contemplated that positioning the reservoir 1 between the aerobars 50 might offer less drag. Accordingly, a keel-shaped reservoir 1 that includes the elements of the reservoir 1 shown in FIGS. 1-4 may be employed. In such an embodiment, due to a lack of space, the straws 10, flow sensors 26, and dual-lumen tubing 23 may be formed integrally with the reservoir 1 and the mount 29 for the CNC 30 may be on the reservoir 1 itself. The fill caps 5 may be located in the portion of the reservoir 1 that extends above the level of the aerobars 50 and is in front of the CNC 30. Both single and dual chamber reservoirs 1 are possible. It will be understood by those skilled in the art that a bracket for mounting the “aerobar reservoir” embodiment may attach to the aerobars 50 themselves. It will be further understood that the reservoir 1 and mount 29 may be made of the same materials as the “downtube reservoir” embodiment.

It should be noted that various changes and modifications to the presently preferred embodiments described herein will be apparent to those skilled in the art. Such changes and modification may be made without departing from the spirit and scope of the present invention and without diminishing its advantages.

I claim:

1. A hydration and nutrition system for monitoring consumption of fluid by a user comprising:
a reservoir adapted to hold a quantity of fluid;
a tube adapted to convey the fluid from the reservoir to the user for consumption;
a flow meter adapted to measure and collect data regarding the flow of fluid from the reservoir through the tube and communicate the data regarding the flow of fluid to a computerized nutritional calculator, wherein the computerized nutritional calculator is adapted to calculate a deficit or surplus of fluid consumed as compared to a consumption plan.

2. The hydration and nutrition system of claim 1 wherein the reservoir is adapted to hold two quantities of fluid in separate reservoirs.

3. The hydration and nutrition system of claim 2 wherein the tube adapted to convey the fluid from the reservoir to the user for consumption is a dual-lumen tube, wherein each lumen in the tube is associated with one of the two reservoirs.

4. The hydration and nutrition system of claim 3 further including a tube extending into each of the reservoirs, wherein each of the tubes extending into the reservoirs connects to a respective lumen of the dual-lumen tube.

5. The hydration and nutrition system of claim 4 wherein each of the tubes extending into each of the reservoirs connects to the respective lumen of the dual-lumen tube through a quick connect.

6. The hydration and nutrition system of claim 5 wherein each of the lumens in the dual-lumen tube includes a bite valve.

7. The hydration and nutrition system of claim 6 wherein each of the bite valves is connected to the dual-lumen tube via a swivel quick connect.

8. The hydration and nutrition system of claim 1 wherein the reservoir includes a fill cap including a pressure relief valve.

9. The hydration and nutrition system of claim 1 wherein the reservoir includes a fill cap including a one-way air entrainment valve.

10. The hydration and nutrition system of claim 1 wherein the reservoir includes a fill cap including a pressure relief valve and a one-way air entrainment valve, wherein the hydration and nutrition system further includes a fill bottle adapted to mate with the fill cap, wherein the fill bottle includes a pressure relief valve and a one-way air entrainment valve.

11. The hydration and nutrition system of claim 1 wherein the computerized nutritional calculator calculates a flow rate of fluid consumed compared to a flow rate in the consumption plan.

12. The hydration and nutrition system of claim 1 wherein the computerized nutritional calculator calculates caloric intake for at least one fluid.

13. The hydration and nutrition system of claim 1 wherein the consumption plan is pre-determined.

14. The hydration and nutrition system of claim 1 wherein the consumption plan is modified by conditions measured over a period of time defining the consumption plan.

15. The hydration and nutrition system of claim 14 wherein the conditions measured over a period of time defining the consumption plan include location and temperature data collected by a location sensor and a temperature sensor associated with the computerized nutritional calculator.

16. The hydration and nutrition system of claim 14 wherein the conditions measured over a period of time defining the consumption plan include data received from bicycle performance sensors.

17. The hydration and nutrition system of claim 1 wherein the computerized nutritional calculator switches from a first consumption plan to a second consumption plan when the computerized nutritional calculator no longer is receiving data from the flow meter.

18. A method of monitoring consumption of fluid by a user during an athletic event including the steps of:
providing a hydration and nutrition system for monitoring consumption of fluid by a user comprising:
a reservoir adapted to hold a quantity of fluid;
a tube adapted to convey the fluid from the reservoir to the user for consumption;
a flow meter adapted to measure and collect data regarding the flow of fluid from the reservoir through the tube and communicate the data regarding the flow of fluid to a computerized nutritional calculator;
providing a consumption plan defining desired replacement of fluid deficits; and
calculating a deficit or surplus of fluid consumed as compared to the consumption plan.

19. The method of claim 18 wherein the consumption plan further defines desired replacement of nutritional deficits.

20. The method of claim 18 wherein the calculation of the deficit includes monitoring of real-time conditions throughout a period of time defining the consumption plan.
